(19) United States
(12) Patent Application Publication
(10) Pub. No.: US 2024/0180571 A1
(43) Pub. Date: Jun. 6, 2024

(54) SMALL VASCULATURE CLOT PINCHING

(71) Applicant: Neuravi Limited, Galway (IE)

(72) Inventors: Anushree DWIVEDI, Galway (IE); Sarah JOHNSON, Galway (IE); Declan LEE, Galway (IE); Karl KEATING, Galway (IE)

(73) Assignee: Neuravi Limited, Galway (IE)

(21) Appl. No.: 18/073,150

(22) Filed: Dec. 1, 2022

(51) Int. Cl. A61B 17/221

(52) U.S. Cl. CPC A61B 17/221; A61B 2017/22079; A61B 2017/2215

(57) ABSTRACT

A clot removal assembly for removing a clot from a vessel that includes a delivery catheter, a clot retrieval device, and a valve assembly is presented herein. In some examples, the clot retrieval device can include a cage structure and a shaft, and the cage structure can be configured to pinch a clot. A portion of the shaft extends through the delivery catheter. In some examples, the valve assembly can include a distal valve, a main body portion, a distal body portion, and a moveable head shell. The distal valve can be configured to engage the proximal region of the delivery catheter. In some examples, the distal body portion can include a plurality of grooves, and the moveable head can be configured to ratchet proximally over the plurality of grooves to thereby maintain tension on the shaft.

### FIELD

The present disclosure generally relates to devices and methods for removing blockages from blood vessels during intravascular medical treatments.

### BACKGROUND

Clot retrieval devices are used in mechanical thrombectomy for endovascular intervention, often in cases where patients are suffering from conditions such as acute ischemic stroke AIS, myocardial infarction MI, and pulmonary embolism PE.

Tough, fibrin rich clots can be located in distal blood vessels that are smaller in comparison to proximal blood vessels. These types of clots can often not be retrieved by aspiration alone, and currently available clot retrieval devices are either too large and damage the vessel walls or too small and not effective at gripping tough distal clots. Making a clot retrieval device of the appropriate size for these tough distal clots has been hitherto unachieved due to difficulties bonding shafts to clot retrieval cage structures. Applicant recognized there is a need to bond a clot retrieval cage structure of an appropriate size for removing these tough distal clots to a shaft that allows use with a catheter of appropriate size for delivery of the cage structure to distal vessels.

The clot may have any of a range of morphologies and consistencies. For example, clot can be difficult to grip, and improper grip can lead to fragmentation which may cause embolization. Compression of a blood clot causes dehydration of the clot and results in a dramatic increase in both clot stiffness and coefficient of friction which necessitates removal by pinching rather than contact aspiration.

Physicians typically rely on visual and tactile feedback to assess clot engagement to a clot retrieval device. In some treatments it can be difficult to determine whether a clot is engaged by a clot retrieval device with the present techniques. It can also be difficult to maintain the appropriate tension between the clot retrieval device and proximal components, but this tension is necessary to maintain engagement with the clot. The devices, systems, and methods disclosed herein.

### SUMMARY

In some examples, a clot removal device is disclosed for removing clot from a vessel. The clot removal device can include a cage structure including a first tapered portion at a proximal end of the cage structure, a shaft including a second tapered portion at a distal end of the shaft, and a proximal bond in which the first tapered portion is positioned in a non-engaging overlap with the second tapered portion. The proximal bond can include a weld between the first tapered portion and the second tapered portion and a material coating over at least a portion of the first tapered portion, at least a portion of the second tapered portion, and at least a portion of the weld.

In some examples, the non-engaging overlap can be laser welded at two points, and the material coating can be bonded to the shaft and the cage structure at two points.

In some examples, the distal shaft portion can be tapered, being narrower distally and wider proximally.

In some examples, the cage structure can further include an open distal end and a proximal spiral section. The proximal spiral section can include a first plurality of struts extending distally from the proximal end of the cage structure and configured to pinch a clot between at least one of the first plurality of struts. The proximal spiral section can extend over a majority of a length of the cage structure from the proximal end to the open distal end.

In some examples, the cage structure can further include a cylindrical body section distal to the proximal spiral section. The cylindrical body section can include a second plurality of struts larger than the first plurality of struts, and the cylindrical body section has a length measuring less than a length of the proximal spiral section. In some examples, the cylindrical body section has an outer diameter of approximately 2.5 mm.

In some examples, a clot removal assembly for removing a clot from a vessel is disclosed. The clot removal assembly can include a delivery catheter, a clot retrieval device, and a valve assembly. In some examples, the delivery catheter can include a distal region and a proximal region. In some examples, the clot retrieval device can include a cage structure and a shaft, and the cage structure can be configured to pinch a clot. A portion of the shaft extends through the delivery catheter. In some examples, the valve assembly can include a distal valve, a main body portion, a distal body portion, and a moveable head shell. The distal valve can be configured to engage the proximal region of the delivery catheter. In some examples, a portion of the shaft extends through a lumen of the main body portion. In some examples, the distal body portion can include a plurality of grooves, and the moveable head can be configured to ratchet proximally over the plurality of grooves to thereby maintain tension on the shaft.

In some examples, the moveable head shell can include a tooth configured to engage the grooves, a threaded portion, and a threaded head configured to engage the threaded portion and crush a seal. The seal, when crushed, locks the shaft in place in relation to the threaded head, and the tooth is configured to disengage with the grooves of the main body upon a flexing away from a central axis of the valve.

In some examples, the cage structure can include a proximal spiral section and a cylindrical body section distal to the proximal spiral section. The cylindrical body section can include a second plurality of struts larger than a first plurality of struts of the proximal spiral section. In some examples, the cylindrical body section can include a length measuring less than a length of the proximal spiral section.

In some examples, the clot removal assembly can further include a proximal bond connecting the shaft to the proximal spiral section. The proximal bond can be formed by laser welding together a non-engaging overlap between a tapered portion of the shaft and a tapered portion of the cage structure and providing a material coating on the proximal bond. In some examples, the non-engaging overlap is laser welded at two points, and the material coating is bonded to the shaft and the cage structure at two points.

In some examples, the moveable head shell can further include external grips disposed on the proximal end of the movable head shell.

In some examples, a distal end of the moveable head shell can include thin plastic cylindrical walls configured to deform responsive to a pinch, and the pinch causes a flexing away from a central axis of the valve.

In some examples, the catheter can further include an inner diameter of approximately 0.013 inches.

In some examples, a method of removing a clot from a blood vessel is disclosed. The method can include deploying at least a part of a cage structure of a clot retrieval device out of a catheter and across the clot, engaging a proximal portion of the catheter to a distal valve of a valve assembly, engaging a proximal portion of the shaft of the clot retrieval device to a moveable head shell of the valve assembly, pulling a moveable head shell of the valve in a proximal direction to pinch the clot, securing the moveable head shell in relation to the distal valve to thereby maintain tension on the shaft, and extracting the cage structure and the clot from the blood vessel.

In some examples, securing the moveable head shell in relation to the distal valve to thereby maintain tension on the shaft can include engaging a tooth disposed on the moveable head shell with a first groove disposed on a distal body portion of the valve assembly and gripping the shaft by compressing a seal between a threaded head and a threaded portion of the moveable head shell.

In some examples, securing the moveable head shell in relation to the distal valve to thereby maintain tension on the shaft can include sliding the moveable head shell proximally and engaging the tooth with a second groove further in a proximal direction than the first.

In some examples, the method can include applying suction to the catheter through a side port disposed on the valve.

In some examples, the method can include releasing the tension by pinching the moveable head shell to disengage the tooth.

In some examples, the method can further include moving the cage structure to pin the clot between the cage structure and an inner wall of a blood vessel.

In some examples, the clot can be made up of a majority of fibrin.

In some examples, the method can include extracting the clot from a distal M2, M3, M4, A2-5, or P2-P5 vessel.

Other aspects and features of the present disclosure will become apparent to those skilled in the pertinent art, upon reviewing the following detailed description in conjunction with the accompanying figures.

### BRIEF DESCRIPTION OF THE DRAWINGS

The above and further aspects of this disclosure are further discussed with the following description of the accompanying drawings, in which like numerals indicate like structural elements and features in various figures. The drawings are not necessarily to scale, emphasis instead being placed upon illustrating principles of the disclosure. The figures depict one or more implementations of the inventive devices, by way of example only, not by way of limitation. It is expected that those of skill in the art can conceive of and combining elements from multiple figures to better suit the needs of the user.

FIG. 1 illustrates a perspective view of a clot removal assembly with a delivery catheter, a clot retrieval device, and a valve assembly according to this disclosure.

FIG. 2 is an isometric view of a clot retrieval device illustrated in FIG. 1.

FIG. 3 illustrates a cross-sectional view of a bond according to this disclosure.

FIG. 4 is an isometric view and a magnified cross-sectional view of a microcatheter according to this disclosure.

FIG. 5A is a cross-sectional view of a blood vessel and a clot and a clot removal assembly disposed therein according to this disclosure.

FIG. 5B is a cross-sectional view of a blood vessel and a clot and a clot removal assembly engaged to the clot according to this disclosure.

FIG. 6 illustrates a valve assembly according to this disclosure.

FIG. 7 illustrates part of a valve assembly including a movable head shell with external grips according to this disclosure.

FIG. 8 is a flow diagram illustrating a method of removing a clot from a blood vessel, according to aspects of the present disclosure.

### DETAILED DESCRIPTION

The following detailed description is merely exemplary in nature and is not intended to limit the disclosure or the application and uses of the disclosure. Although the description of the disclosure is in many cases in the context of treatment of intracranial arteries, the disclosure may also be used in other body passageways as previously described.

The terms “distal” or “proximal” are used in the following description with respect to a position or direction relative to the treating physician. “Distal” or “distally” are a position distant from or in a direction away from the physician. “Proximal” or “proximally” or “proximate” are a position near or in a direction toward the physician.

As discussed herein, a “patient” or “subject” can be a human or any animal. It should be appreciated that an animal can be a variety of any applicable type, including, but not limited to, mammal, veterinarian animal, livestock animal or pet-type animal, etc. As an example, the animal can be a laboratory animal specifically selected to have certain characteristics similar to a human e.g., rat, dog, pig, monkey, or the like.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values+20% of the recited value, e.g., “about 90%” may refer to the range of values from 71% to 99%.

When used herein, the terms “tubular” and “tube” are to be construed broadly and are not limited to a structure that is a right cylinder or strictly circumferential in cross-section or of a uniform cross-section throughout its length. For example, the tubular structure or system is generally illustrated as a substantially right cylindrical structure. However, the tubular system may have a tapered or curved outer surface without departing from the scope of the present invention.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

It must also be noted that, as used in the specification and the appended claims, the singular forms “a,” “an,” and “the” include plural referents unless the context clearly dictates otherwise. Ranges can be expressed herein as from “about” or “approximately” one particular value and/or to “about” or “approximately” another particular value. When such a range is expressed, other exemplary examples include from the one particular value and/or to the other particular value.

Accessing cerebral, coronary and pulmonary vessels involves the use of a number of commercially available products and conventional procedural steps. Access products such as guidewires, guide catheters, angiographic catheters and microcatheters are described elsewhere and are regularly used in catheter lab procedures. It is assumed in the descriptions below that these products and methods are employed in conjunction with the device and methods of this disclosure and do not need to be described in detail.

A common theme across many of the disclosed designs is a clot removal assembly 200 for removing a clot 700 from a vessel. The clot removal assembly 200 can include a delivery catheter 300, a clot retrieval device, and a valve assembly.

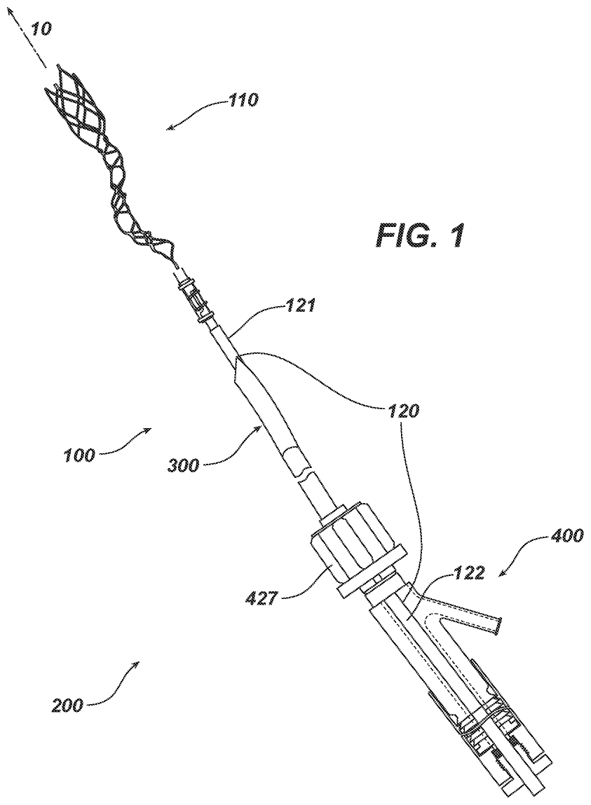

FIG. 1 is an illustration of one example clot removal assembly for removing a clot 700 from a vessel. The clot removal assembly can include a delivery catheter 300, a clot retrieval device 110, and a valve assembly 400. In some examples, the clot retrieval device 100 can include a cage structure 110 and a shaft 120, and the cage structure 110 can be configured to pinch a clot 700. A portion of the shaft 120 extends through the delivery catheter 300. In some examples, the valve assembly 400 can include a distal valve 427, a main body portion 410, a distal body portion 412, and a moveable head shell 420. The distal valve 427 can be configured to engage the proximal region 320 of the delivery catheter 300. In some examples, a portion of the shaft 120 extends through a lumen of the main body portion. In some examples, the distal body portion 412 can include a plurality of grooves 411, and the moveable head 420 can be configured to ratchet proximally over the plurality of grooves 411 to thereby maintain tension on the shaft 120. In some examples, the shaft 120 may be a tapered wire shaft, and may be made of stainless steel, MP35N, Nitinol or other material of a suitably high modulus and tensile strength.

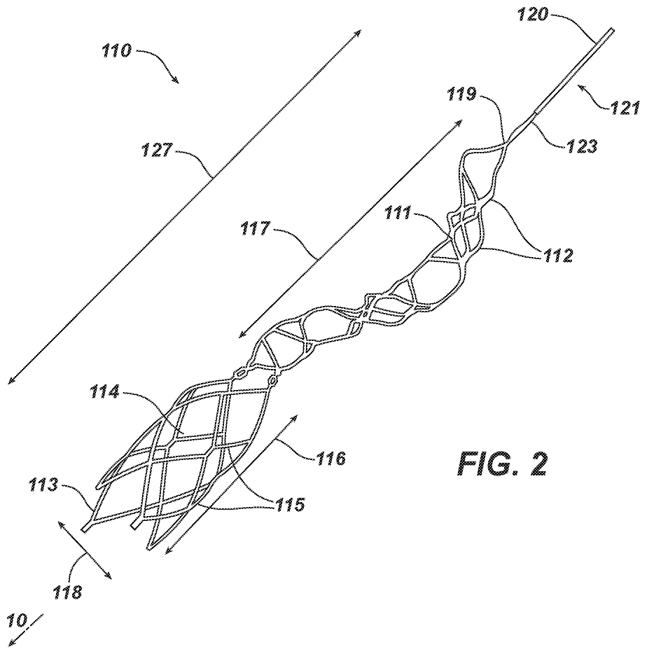

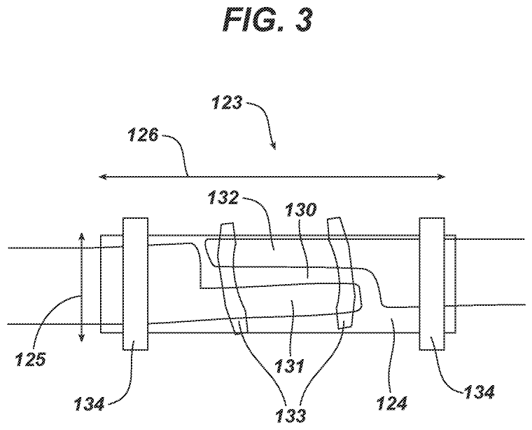

FIG. 2 is an isometric view of a clot retrieval device illustrated in FIG. 1. In some examples, the clot removal device 100 can include a cage structure 110 including a first tapered portion at a proximal end 119 of the cage structure 110, a shaft 120 including a second tapered portion at a distal end of the shaft 120, and a proximal bond 123 in which the first tapered portion is positioned in a non-engaging overlap 130 with the second tapered portion (scc FIG. 3).

In some examples, the cage structure 110 can further include an open distal end 113 and a proximal spiral section 111. The proximal spiral section 111 can include a first plurality of struts 112 extending distally from the proximal end 119 of the cage structure 110 and configured to pinch a clot 700 between at least one of the first plurality of struts 112. The proximal spiral section 111 can extend over a majority of a length 127 of the cage structure 110 from the proximal end 119 to the open distal end 113.

In some examples, the cage structure 110 can further include a cylindrical body section 114 distal to the proximal spiral section 111. The cylindrical body section 114 can include a second plurality of struts 115 larger than the first plurality of struts 112, and the cylindrical body section 114 has a length 116 measuring less than a length 117 of the proximal spiral section 111. In some examples, the cylindrical body section 114 has an outer diameter 118 of approximately 2.5 mm.

The cage structures 110 of the designs disclosed are desirably made from a material capable of recovering its shape automatically once released from a highly strained delivery configuration. A superelastic material such as Nitinol or an alloy of similar properties is particularly suitable. The material can be in many forms such as wire or strip or sheet or tube. A particularly suitable manufacturing process is to laser cut a Nitinol tube and then heat set and electropolish the resultant structure to create a framework of struts and connecting elements. This framework can be any of a huge range of shapes as disclosed herein and may be rendered visible under fluoroscopy through the addition of alloying elements or through a variety of other coatings or marker bands.

In some examples, the cage structure 110 can further include cells, struts, and a variety of shapes and designs configured for pinching fibrin rich clots, including those described in U.S. Pat. Nos. 10,292,723; 10,363,054; 10,617,435; 11,253,278; and 11,147,572, each of which are incorporated by reference in their entirety as if set forth verbatim herein. Compression of the clot 700 by the cage structure 110, including by the cylindrical body section 114, can alter the clot properties and make the clot 700 less amenable to retrieval by making it firmer and “stickier” similar to as described in WO2012/120490A, the entire contents of which are herein incorporated by reference.

In some examples, distal advancement of the catheter 300 relative to the cage structure 110 can compress the clot 300 between the distal end of the catheter 300 and the cage structure 110, increasing the pinch on the clot 700 and the security of the trapped clot segment. The user may feel this pinch as a resistance and stop advancing the catheter 300, the user may advance the delivery catheter 300 a fixed distance over the shaft 120 for example 30% to 50% of the shaft length.

FIG. 3 illustrates a cross-sectional view of the bond according to this disclosure. In some examples, the proximal bond 123 can include a weld between the first tapered portion and the second tapered portion and a material coating 124 over at least a portion of the first tapered portion, at least a portion of the second tapered portion, and at least a portion of the weld. In some examples, the non-engaging overlap 130 can be laser welded at two points 133, and the material coating 124 can be bonded to the shaft 120 and the cage structure 110 at two points 134.

In some examples, the clot removal assembly can further include a proximal bond 123 connecting the shaft 120 to the proximal spiral section 111. The proximal bond 123 can be formed by laser welding together a non-engaging overlap 130 between a tapered portion of the shaft 120 and a tapered portion of the cage structure 110 and providing a material coating 124 on and/or over the proximal bond 123. In some examples, the non-engaging overlap 130 is laser welded 133 at two points, and the material coating 124 is bonded to the shaft 120 and the cage structure 110 between two points 134. Thus, in one example, once the non-engaging overlap 130 is welded 133, the material coating 124 can act as a “sleeve” further covering and reinforcing the joint (i.e. the non-engaging overlap 130) and is coated where necessary (i.e. between points 134) to provide that support. Note that the material coating 124 can be a physical polymer sleeve in some examples.

One object of this invention is to reduce the size of the proximal bond 123, which acts as a joint between the cage structure 110 and shaft 120. Prior art joints can be 0.018″ at the widest point. The present proximal bond 123 allows for a reduced size to pass through a 0.013″ delivery catheter. Even at the maximum size, this can be an approximately 38% reduction in size as compared to the prior art. Note that certain prior art stent retrievers are sized to treat large vessel occlusions (LVO), which occur when a major artery in the brain is blocked. An LVO stroke blockage occurs in one of the following major cerebral vessels in the brain: Internal Carotid Artery (ICA) ICA terminus (T-lesion; T occlusion). More distal clots form in the smaller arteries, including in the distal M2, M3, M4, A2-A5, P2-P5 vessels. The smaller the proximal bond 123, the smaller the delivery catheter and thus the cage device 110 can be delivered farther into cerebral vascular than most prior art devices.

In some examples, the distal shaft portion 121 can be tapered, being narrower distally and wider proximally.

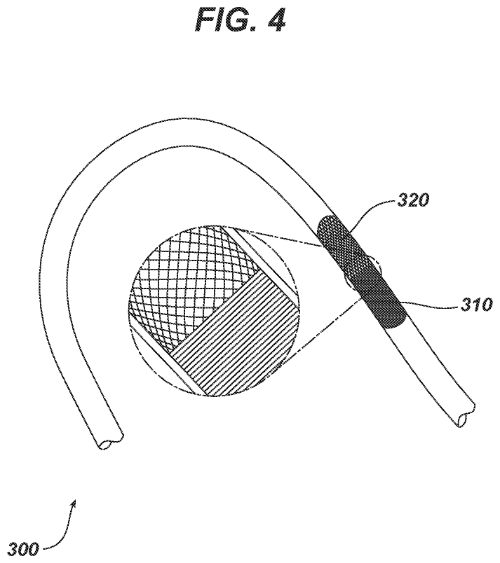

FIG. 4 is an isometric view and a magnified cross-sectional view of an example catheter 300. In some examples, the delivery catheter 300 can include a distal region 310 and a proximal region 320. In some examples, the distal portion 310 of the delivery catheter 300 can include a coiled core and the proximal portion 320 of the delivery catheter 300 can include a braided core.

In some examples, the coiled core can provide the catheter 300 with a level of flexibility that allows the user to better navigate tortuous distal vessels. In some examples, the braided core of the proximal portion 320 can provide the delivery catheter 300 with a level of column strength that aids the user in navigating the tip of the microcatheter to the target anatomy.

In some examples, the catheter 300 can be configured such that a compressive force applied to a majority of the length of the catheter 300 results in the proximal region 320 remaining substantially straight and the distal region becoming wavy.

In some examples, the catheter 300 can be configured to deliver a clot retrieval device 100 across a clot 700.

In some examples, the catheter 300 can be configured to pinch the clot 700 between a distal end of the catheter and the clot retrieval device 100.

In some examples, the catheter 300 can further include an inner diameter of approximately 0.013 inches.

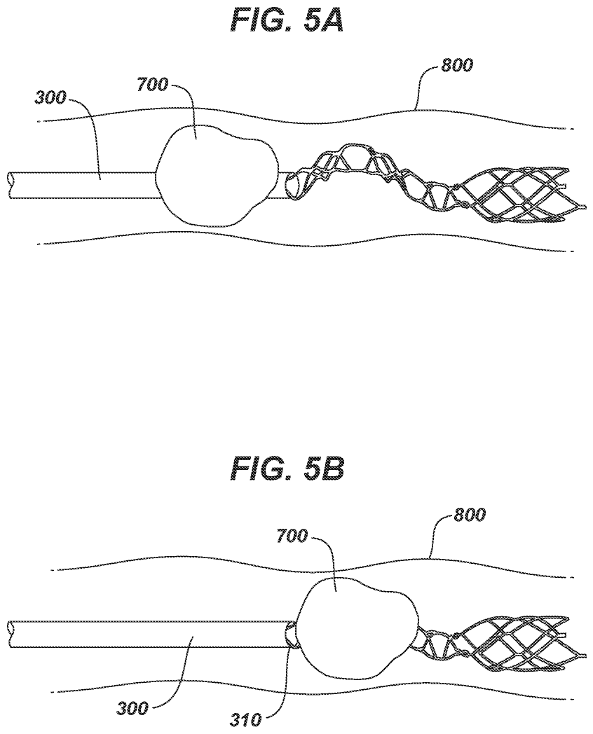

FIG. 5A is a cross-sectional view of a blood vessel 800, a clot 700, and a clot removal assembly 200 disposed therein. In some examples, the catheter 300 can be configured to deliver the clot retrieval device 100 across the clot 700.

FIG. 5B is a cross-sectional view of the blood vessel 800, the clot 700, and the clot removal assembly 200 illustrated in FIG. 1 with the clot removal assembly 200 engaged to the clot 300. In some embodiments, the proximal spiral section 111 of the cage structure 110 can be sized to wind within the blood vessel 800 against walls of the blood vessel 800. The spiral section 111 can push the clot 700 into the blood vessel wall, pinning the clot 700 so that the clot 700 is inhibited from moving distally through the vessel 800. The catheter 300 can be advanced distally, causing the spiral section 111 to move toward each other, like tweezers.

The clot 700 can have a firm portion that can be pinched between the tip of the catheter 300 and the spiral section 111 of the cage structure 110. The pinch can be achieved by forwarding the catheter 300 or intermediate catheter over the cage structure 110 until a portion of the clot 700 is compressed between the tip of the catheter 300 and a crown or strut on the proximal portion of the cage structure 110. This pinch facilitates removal of the clot 700 as it increases the grip of the cage structure 110 on the clot, particularly fibrin rich clots. Proximal retraction of the pinched clot may elongate the clot 700 reducing the dislodgement force by pulling the clot 700 away from the vessel wall during the dislodgement process.

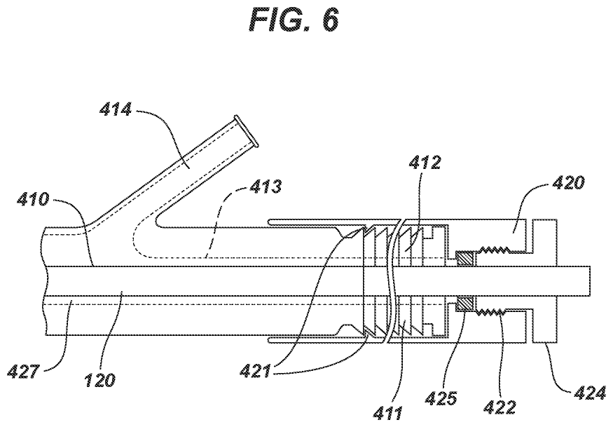

FIG. 6 is a cross-section of a valve assembly 400 according to this disclosure. In some examples, the valve assembly 400 can include a distal valve 427, a main body portion 410, a distal body portion 412, and a moveable head shell 420. The distal valve 427 can be configured to engage the proximal region 310 of the delivery catheter 300. In some examples, a portion of the shaft 120 extends through a lumen 413 of the main body portion 410. In some examples, the distal body portion 412 can include a plurality of grooves 411, and the moveable head shell 420 can be configured to ratchet proximally over the plurality of grooves 411 to thereby maintain tension on the shaft 120.

Shaft tension can be important during certain procedures. As noted above, the present cage structure 110 is designed to pinch small, tough clots. Once the clot is pinched, any release of the tension on the shaft can release the pinch and clot. Allowing the user to maintain tension on the shaft by providing controls on the valve assembly 400 helps facilitate maintaining that tension.

In some examples, the moveable head shell 420 can include a tooth 421 configured to engage the grooves 411, a threaded portion 422, and a threaded head 424 configured to engage the threaded portion 422 and crush a seal 425, wherein the seal 425 when crushed locks the shaft 120 in place in relation to the threaded head 424, and wherein the tooth 421 is configured to disengage with the grooves 411 of the main body 410 upon a flexing away from a central axis 430 of the valve 400.

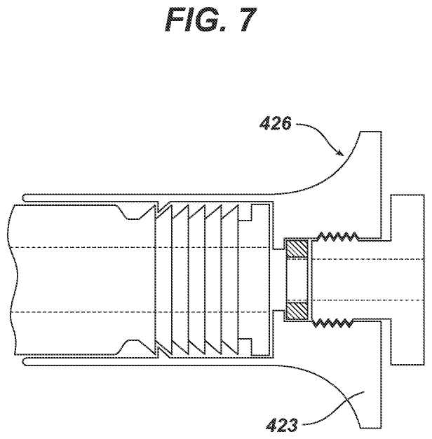

FIG. 7 illustrates part of a valve assembly 400 including a movable head shell 420 with external grips 426 according to this disclosure. In some examples, the moveable head shell 420 can further include external grips 426 disposed on the proximal end 423 of the movable head shell 420. In some examples, a distal end 427 of the moveable head shell 420 can include thin plastic cylindrical walls configured to deform responsive to a pinch, and wherein the pinch causes the flexing away from the central axis of the valve.

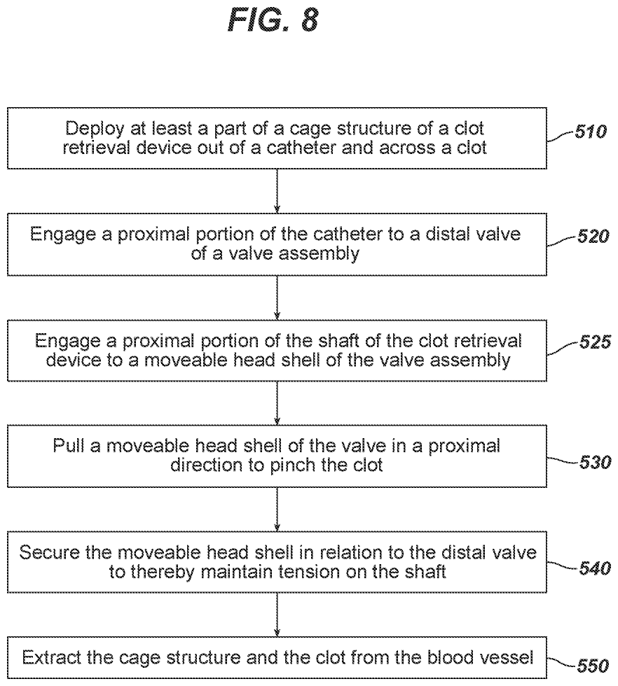

FIG. 8 shows a method 500 of removing a clot from a blood vessel as disclosed herein. The method steps in FIG. 8 can be implemented by any of the example means described herein or by similar means, as will be appreciated.

At block 510, method 500 can include deploying at least a part of a cage structure of a clot retrieval device out of a catheter and across the clot. The cage structure of the clot removal device can be configured similarly to an example cage structure 110 of the clot removal device 100 disclosed herein, variations thereof, and alternatives thereto as understood by a person skilled in the pertinent art. The cage structure can be deployed similarly to as illustrated in FIG. 5A and FIG. 5B and as otherwise disclosed herein, variations thereof, and alternatives thereto as understood by a person skilled in the pertinent art. The clot removal device can be configured similarly to an example clot removal device 100 disclosed herein, variations thereof, and alternatives thereto as understood by a person skilled in the pertinent art.

At block 520, the method 500 can include engaging a proximal portion of the catheter to a distal valve of a valve assembly. The catheter can be configured similarly to an example catheter 300 disclosed herein, variations thereof, and alternatives thereto as understood by a person skilled in the pertinent art. The valve assembly can be configured similarly to an example valve assembly 400 (and subcomponents thereof) disclosed herein, variations thereof, and alternatives thereto as understood by a person skilled in the pertinent art.

At block 525, the method 500 can include engaging a proximal portion of the shaft of the clot retrieval device to a moveable head shell of the valve assembly. The shaft can be configured similarly to an example shaft 120 disclosed herein, variations thereof, and alternatives thereto as understood by a person skilled in the pertinent art.

At block 530, the method 500 can include pulling the moveable head shell of the valve in a proximal direction to pinch the clot.

At block 540, the method 500 can include securing the moveable head shell in relation to the distal valve to thereby maintain tension on the shaft.

At block 550, the method 500 can include extracting the cage structure and the clot from the blood vessel. In some examples, the method 500 can include extracting the clot 700 from a distal M2, M3, M4, A2-5, or P2-P5 vessel. The size of any of these vessels is known in the art and it will be appreciated by those skilled in the pertinent art that components of the clot removal assembly can be sized appropriately to achieve the goal of removing the clot from these vessels. Despite this knowledge of the vessel sizes being known by those skilled in the pertinent art, it has been hitherto very difficult to make a clot removal device as disclosed herein due to difficulties coupling the pinching cage structure to the shaft. This problem is solved by the bond described herein.

In some examples, the method 500 can include using fluoroscopic imaging techniques.

In some examples, the clot 700 can be made up of a majority of fibrin.

A range of designs are envisaged for each of these elements as described throughout this document, and it is intended that any of these elements can be used in conjunction with any other element, although to avoid repetition they are not shown in every possible combination.

Other aspects and features of the present disclosure will become apparent to those of ordinary skill in the art, upon reviewing the following detailed description in conjunction with the accompanying figures.

In describing examples, terminology is resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method can be performed in a different order than those described herein without departing from the scope of the disclosed technology. Similarly, it is also to be understood that the mention of one or more components in a device, system, or assembly does not preclude the presence of additional components or intervening components between those components expressly identified.

The descriptions contained herein are examples of the disclosure and are not intended in any way to limit the scope of the disclosure. While particular examples of the present disclosure are described, various modifications to devices and methods can be made without departing from the scope and spirit of the disclosure. For example, while the examples described herein refer to particular components, the disclosure includes other examples utilizing various combinations of components to achieve a described functionality, utilizing alternative materials to achieve a described functionality, combining components from the various examples, combining components from the various example with known components, etc. The disclosure contemplates substitutions of component parts illustrated herein with other well-known and commercially available products. To those having ordinary skill in the art to which this disclosure relates, these modifications are often apparent and are intended to be within the scope of the claims which follow.

What is claimed is:

1. A clot removal device comprising:
a cage structure comprising a first tapered portion at a proximal end of the cage structure;
a shaft comprising a second tapered portion at a distal end of the shaft; and
a proximal bond in which the first tapered portion is positioned in a non-engaging overlap with the second tapered portion, the proximal bond comprising a weld between the first tapered portion and the second tapered portion and a material coating over at least a portion of the first tapered portion, at least a portion of the second tapered portion, and at least a portion of the weld.

2. The clot removal device of claim 1, wherein the non-engaging overlap is laser welded at two points, and
wherein the material coating is bonded to the shaft and the cage structure at two points.

3. The clot removal device of claim 1, wherein the second tapered portion is tapered, being narrower distally and wider proximally.

4. The clot removal device of claim 1, wherein the cage structure further comprises:
an open distal end; and
a proximal spiral section comprising a first plurality of struts extending distally from the proximal end of the cage structure and configured to pinch a clot between at least one of the first plurality of struts, the proximal spiral section extending over a majority of a length of the cage structure from the proximal end to the open distal end.

5. The clot removal device of claim 4, wherein the cage structure further comprises a cylindrical body section distal to the proximal spiral section,
wherein the cylindrical body section comprises a second plurality of struts larger than the first plurality of struts, and
wherein the cylindrical body section comprises a length measuring less than a length of the proximal spiral section.

6. The clot removal device of claim 5, wherein the cylindrical body section comprises an outer diameter of approximately 2.5 mm.

7. A clot removal assembly comprising:
a delivery catheter comprising a distal region and a proximal region;
a clot retrieval device comprising a cage structure and a shaft, wherein the cage structure is configured to pinch a clot, and wherein a portion of the shaft extends through the delivery catheter; and
a valve assembly comprising a distal valve, a main body portion, a distal body portion, and a moveable head shell, wherein the distal valve is configured to engage the proximal region of the delivery catheter, wherein a portion of the shaft extends through a lumen of the main body portion, wherein the distal body portion comprises a plurality of grooves, and wherein the moveable head shell is configured to ratchet proximally over the plurality of grooves to thereby maintain tension on the shaft.

8. The clot removal assembly of claim 7, wherein the moveable head shell comprises:
a tooth configured to engage the grooves;
a threaded portion; and
a threaded head configured to engage the threaded portion and crush a seal,
wherein the seal when crushed locks the shaft in place in relation to the threaded head, and
wherein the tooth is configured to disengage with the grooves of the main body portion upon a flexing away from a central axis of the valve assembly.

9. The clot removal assembly of claim 7, wherein the cage structure comprises a proximal spiral section and a cylindrical body section distal to the proximal spiral section, wherein the cylindrical body section comprises a second plurality of struts larger than a first plurality of struts of the proximal spiral section.

10. The clot removal assembly of claim 9, wherein the cylindrical body section comprises a length measuring less than a length of the proximal spiral section.

11. The clot removal assembly of claim 9 further comprising a proximal bond connecting the shaft to the proximal spiral section, wherein the proximal bond is formed by laser welding together a non-engaging overlap between a tapered portion of the shaft and a tapered portion of the cage structure and providing a material coating on the proximal bond.

12. The clot removal assembly of claim 11, wherein the non-engaging overlap is laser welded at two points, and
wherein the material coating is bonded to the shaft and the cage structure at two points.

13. The clot removal assembly of claim 7, wherein the moveable head shell further comprises external grips disposed on a proximal end of the moveable head shell.

14. The clot removal assembly of claim 7, wherein a distal end of the moveable head shell comprises thin plastic cylindrical walls configured to deform responsive to a pinch, and wherein the pinch causes the flexing.

15. The clot removal assembly of claim 14, wherein the delivery catheter further comprises an inner diameter of approximately 0.013 inches.

16. A method of removing a clot from a blood vessel comprising:
deploying at least a part of a cage structure of a clot retrieval device out of a catheter and across the clot;
engaging a proximal portion of the catheter to a distal valve of a valve assembly;
engaging a proximal portion of a shaft of the clot retrieval device to a moveable head shell of the valve assembly;
pulling a moveable head shell of the valve in a proximal direction to pinch the clot;
securing the moveable head shell in relation to the distal valve to thereby maintain tension on the shaft; and
extracting the cage structure and the clot from the blood vessel.

17. The method of claim 16, wherein securing the moveable head shell in relation to the distal valve to thereby maintain tension on the shaft comprises:
engaging a tooth disposed on the moveable head shell with a first groove disposed on a distal body portion of the valve assembly; and
gripping the shaft by compressing a seal between a threaded head and a threaded portion of the moveable head shell.

18. The method of claim 17, wherein securing the moveable head shell in relation to the distal valve to thereby maintain tension on the shaft comprises sliding the moveable head shell proximally and engaging the tooth with a second groove further in a proximal direction than the first groove.

19. The method of claim 18, further comprising applying suction to the catheter through a side port disposed on the valve.

20. The method of claim 18, further comprising releasing the tension by pinching the moveable head shell to disengage the tooth.
